(19) United States
(12) Patent Application Publication
(10) Pub. No.: US 2009/0069604 A1
(43) Pub. Date: Mar. 12, 2009

(54) PROCESS FOR HYDROGENATING METHYLOLALKANALS

(75) Inventors: Steffen Maas, Bubenheim (DE); Thorsten Johann, Ludwigshafen (DE); Michael Koch, Mannheim (DE); Tilman Sirch, Schifferstadt (DE); Stephan Schlitter, Limburgerhof (DE); Stefan Rittinger, Mannheim (DE); Michael Steiniger, Neustadt (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/279,792

(22) PCT Filed: Feb. 23, 2007
(86) PCT No.: PCT/EP2007/051760

§ 371 Date: Aug. 18, 2008

(30) Foreign Application Priority Data
Mar. 1, 2006 (DE) 10 2006 009 838.2

(51) Int. Cl. C07C 45/62

(52) U.S. Cl. 568/465

(57) ABSTRACT

A process for catalytically hydrogenating methylolalkanals of the general formula

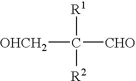

in which R¹ and R² are each independently a further methylol group or an alkyl group having from 1 to 22 carbon atoms or an aryl or aralkyl group having from 6 to 33 carbon atoms, in the liquid phase over a hydrogenation catalyst, which comprises setting a pH of from 7.0 to 9.0 in the hydrogenation effluent by adding at least one tertiary amine, an inorganic base or an inorganic or organic acid to the hydrogenation feed.

The invention relates to a process for catalytically hydrogenating methylolalkanals in the liquid phase over a hydrogenation catalyst by pH control of the hydrogenation effluent.

The catalytic hydrogenation of carbonyl compounds, for example aldehydes, to prepare simple and functionalized alcohols is assuming an important position in the production streams of the commodity chemicals industry. This is particularly true of the hydrogenation of aidehydes which are obtainable via the oxo process or aldol reaction.

Methylolalkanals are obtainable by aldol reaction of alkanals with excess formaldehyde in the presence of stoichiometric amounts of base. WO 01/51438 discloses the use of inorganic hydroxides such as sodium hydroxide or calcium hydroxide as the base. WO 98/28253 and DE-A1957591 describe amines as basic catalysts for aldolization, and WO 98/29374 basic ion exchangers. The methylolalkanal is obtained by these processes as a from 20 to 80% by weight aqueous solution. The pH of this aqueous solution is only from 3.5 to 6.0, since the basic catalyst of the aldolization also catalyzes the Cannizzaro reaction of the formaldehyde to give formic acid which is in turn neutralized at least partly by the base.

When the intention is to prepare polyhydric alcohols such as pentaerythritol, neopentyl glycol or trimethylolpropane from aqueous methylolalkanal solutions, these solutions have to be hydrogenated.

This hydrogenation is generally carried out at temperatures of above 80° C. Dissociations of the methylol group to give free aldehyde, the Cannizzaro reaction of the formaldehyde to give formic acid and additionally ether, ester and acetal formation are observed in the hydrogenation reactor. These side reactions lead to low hydrogenation selectivities and to low yields of polyhydric alcohol.

In addition, many hydrogenation catalysts are not stable under these conditions. Especially catalysts based on the oxides of aluminum and silicon, as known from EP-A 44 444 and WO 95/32171, lose activity in the presence of these aqueous methylolalkanal solutions under hydrogenation conditions, and experience has shown that this leads to a distinctly lower conversion over a period of a few months.

On the industrial scale, it would be possible to at least partly compensate for this by increasing the hydrogenation temperature stepwise. In addition to the uneconomic increased energy consumption that this measure requires, side reactions secondly increase greatly from a certain temperature and lead to increased use numbers (consumption of feedstocks) or to less pure product, so that the catalyst has to be replaced by a new one.

For example, in the hydrogenation of hydroxypivalaldehyde or of dimethylolbutanal to the corresponding alcohols neopentyl glycol (NPG) and trimethylolpropane (TMP), a retro-aldol reaction takes place with increasing temperature. The aidehydes formed are hydrogenated to undesired by-products (in the case of NPG preparation, isobutanol and methanol are thus formed; in the case of TMP preparation, 2-methylbutanol, 2-ethyl-1,3-propanediol and methanol), and the yield is reduced correspondingly. In the case of NPG synthesis, the formation of the cyclic acetal of NPG and hydroxypivalaldehyde (HPA) is also observed to an increased extent at elevated temperature. This by-product cannot be separated from NPG by distillation and therefore leads to a less pure product of value. Moreover, high temperatures promote the thermal Tischchenko reaction of HPA to give neopentyl glycol hydroxypivalate (NHP). This partly hydrolyzed to NPG and hydroxypivalic acid (HPA), which leads in turn to a lowering of the pH. Owing to these side reactions, temperature increase as a means of keeping the hydrogenation activity of an aging catalyst constant is limited by economic factors such as yield and product purity.

According to the invention, it has now been found that the pH in the hydrogenation reactor has a crucial influence on the hydrogenation result and the catalyst activity. The pH in the hydrogenation reactor is determined crucially by its formic acid content. Moreover, the hydrolysis of NHP to NPG and HPA also has an influence on the pH.

Formic acid which has been formed in the aldolization as a by-product via a Cannizzaro reaction from formaldehyde is decomposed in the course of the industrial scale hydrogenation to CO₂ and H₂ or to CO and H₂O. CO and CO₂ can be detected in the offgas of the hydrogenation. In addition to the temperature, the decomposition rate of the undesired formic acid by-product depends crucially upon the age of the catalyst. With increasing age of the catalyst, the decomposition rate of formic acid under constant reaction conditions also decreases permanently.

In general, the hydrogenation reactor is operated on the industrial scale, in order to ensure good removal of the heat of hydrogenation, with a high circulation ratio, i.e. the amount circulated is selected to be greater than the amount of fresh feed (supply of fresh feed). The pH in the reactor therefore corresponds to the pH of the hydrogenation effluent, but is significantly higher than the pH of the hydrogenation feed. The difference in the pH between feed and effluent is determined by the activity of the catalyst with regard to the decomposition of formic acid, and also by temperature, amount of offgas and loading.

Processes are already known from the prior art in which attempts have been made to influence the hydrogenation result via the pH.

PCT/WO 2004/092097 describes a hydrogenation process in which the hydrogenation feed is neutralized by adding a base, with the aim of preventing adverse effects of the pH on the mechanical stability of the catalyst support. A disadvantage of this process is that the setting of a pH in the hydrogenation feed on the basis of the above-described effects such as the decomposition of formic acid or the formation of hydroxypivalic acid does not enable effective pH control in the hydrogenation reactor. The fluctuations in the pH in the hydrogenation reactor which necessarily occur in this method prevent optimal hydrogenation conversion and the achievement of optimal selectivity.

JP 2004-182622 describes a hydrogenation process in which the pH is adjusted to pH 5.5-7.5 in the hydrogenation feed. At smaller pH, discharge of the active metal from the catalyst was observed, which led to a continuous activity loss. Moreover, the metal traces interfere in the further workup. In the case of relatively high pH values, aldol condensations were observed, which reduce the selectivity of the process.

It was therefore an object of the invention to provide a process for catalytically hydrogenating methylolalkanals, in which polyhydric alcohols with good hydrogenation selectivities and yields can be made obtainable at high lifetimes of the catalyst.

This object is achieved by a process for catalytically hydrogenating methylolalkanals of the general formula

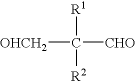

in which R¹ and R² are each independently a further methylol group or an alkyl group having from 1 to 22 carbon atoms or an aryl or aralkyl group having from 6 to 33 carbon atoms, in the liquid phase over a hydrogenation catalyst, which comprises setting a pH of from 7.0 to 9.0 in the hydrogenation effluent by adding at least one tertiary amine, an inorganic base or an inorganic or organic acid to the hydrogenation feed.

The process according to the invention enables effective pH control in the reactor by setting the inventive pH range in the hydrogenation effluent. pH variations as a result of side reactions of the hydrogenation and the influence of an aging catalyst can be avoided. High conversions, selectivities and lifetimes of the catalyst are achieved.

In this application, hydrogenation feed is understood to mean an aqueous solution comprising methylolalkanal of the general formula I, in particular an aqueous solution comprising from 20 to 80% by weight of methylolalkanal. Such a hydrogenation feed is preferably prepared according to WO 98/28253 or DE-A 1 957 591 by reacting aldehydes with formaldehyde.

The procedure is to react the aldehyde with from 1 to 8 times the amount of formaldehyde in the presence of a tertiary amine (aldolization) and to separate the reaction mixture thus obtained into two solutions, one solution comprising the methylolalkanal mentioned and the other solution unconverted starting material. This last solution is recycled into the reaction. The separation is effected by distillation or simple removal of the aqueous phase from the organic phase. The aqueous solution comprising the methylolalkanal can be used as the hydrogenation feed in the process according to the invention.

However, it is also possible to prepare the aqueous methylolalkanal solution as a hydrogenation feed by other prior art processes, for example by the processes known from WO 01/51438, WO 97/17313 and WO 98/29374.

In a preferred variant of the process according to the invention, a particularly low-formaldehyde or formaldehyde-free aqueous methylolalkanal solution is used as the hydrogenation feed. In a low-formaldehyde methylolalkanal solution, the content of formaldehyde is below 5% by weight. The removal of formaldehyde from the aldolization effluent, which has been obtained, for example, according to WO 98/28253 can be effected by processes known from the prior art, for example by distillation.

The methylolalkanal of the general formula I is preferably dimethyloialkanal, pentaerythrose or hydroxypivalaldehyde.

Upstream of the hydrogenation reactor entrance, the hydrogenation feed is mixed with tertiary amine, inorganic base or inorganic or organic acid until the hydrogenation effluent which is withdrawn downstream of the reactor exit has a pH of from 7.0 to 9.0, for the preparation of neopentyl glycol preferably a pH of from 8.0 to 9.0, for the preparation of trimethylolpropane preferably a pH of from 7.0 to 8.0. It is also possible to feed the hydrogenation feed and the tertiary amine, the inorganic base or the inorganic or organic acid separately into the reactor and to mix them there.

Examples of suitable tertiary amines include the amines listed in DE-A 25 07 461. Preferred tertiary amines are tri-n-C₁- to C₄-alkylamines, and particular preference is given to trimethylamine, triethylamine, tri-n-propylamine and tri-n-butylamine. In general, up to 10% by weight (based on the hydrogenation feed) of the tertiary amine is added for pH control in the process according to the invention. The amine may be used as the pure substance or as an aqueous solution.

Suitable inorganic bases are the carbonates, hydrogencarbonates and hydroxides of the alkali metals and alkaline earth metals.

Amines are to be used particularly advantageously for pH adjustment, since they form thermally decomposable salts, which can be dissociated again after the hydrogenation, with formic acid. Thus, a salt burden can be prevented and the tertiary amine can be recycled into the process.

The use of the same tertiary amine in the aldolization process to give the methylolalkanal the condensation of higher aidehyde and formaldehyde—and in the hydrogenation is particularly advantageous.

According to the invention, the inorganic or organic acids used may be mineral acids such as hydrochloric acid, sulfuric acid or phosphoric acid, or organic acids such as citric acid, acetic acid or ethylhexanoic acid. Preference is given to using acetic acid. In general, 0 and 3% by weight (based on the hydrogenation feed) of a 10% aqueous solution of the acid are added for pH control.

The pH is measured with the known techniques, preferably with a glass electrode and a pH meter.

Catalysts usable in accordance with the invention are catalysts suitable for hydrogenations which preferably have at least one metal of transition group 8 to 12 of the Periodic Table of the Elements, such as Fe, Ru, Os, Co, Rh, Ir, Ni, Pd, Pt, Cu, Ag, Au, Zn, Cd, Hg, preferably Fe, Co, Ni, Cu, Ru, Pd, Pt, more preferably Cu, preferably on a customary support material, more preferably on a support material from the oxides of titanium, zirconium, hafnium, silicon and/or aluminum. The catalysts usable in accordance with the invention can be prepared by processes known from the prior art for preparing such supported catalysts. Supported catalysts which comprise copper on an alumina or titania support material in the presence or absence of one or more of the elements magnesium, barium, zinc or chromium may also be used with preference. Such catalysts and their preparation are known from WO 99/44974.

Further suitable catalysts for the inventive hydrogenation are supported copper catalysts as described, for example, in WO 95/32171, and the catalysts disclosed in EP-A 44 444 and DE 19 57 591.

The hydrogenation can be carried out batchwise or continuously, for example in a reactor tube filled with a catalyst bed, in which the reaction solution is passed over the catalyst bed, for example in trickle mode or liquid phase mode, as described in DE-A 1 941 633 or DE-A 2 040 501. It may be advantageous to recycle a substream of the reaction effluent, if appropriate with cooling, and to pass it back through the fixed catalyst bed. This circulation mode is preferably operated with a ratio of circulation to feed of from 10:1 to 20:1. It may likewise be advantageous to carry out the hydrogenation in a plurality of reactors connected in series, for example in from 2 to 4 reactors, in which case the hydrogenation reaction is carried out in the individual reactors before the last reactor only up to a partial conversion of, for example, from 50 to 98%, and the hydrogenation is completed only in the last reactor. It may be appropriate to cool the hydrogenation effluent from the preceding reactor before it enters the next reactor, for example by means of cooling apparatus or by injecting cold gases such as hydrogen or nitrogen, or introducing a substream of cold reaction solution.

The hydrogenation temperature is generally between 50 and 180° C., preferably 90 and 140° C. The hydrogenation pressure employed is generally from 10 to 250 bar, preferably from 20 to 120 bar.

The hydrogenation can be carried out with addition of an inert solvent. Usable solvents are water, cyclic ethers such as THF or dioxane, and also acyclic ethers, as are lower alcohols, for example methanol, ethanol or 2-ethylhexanol.

Otherwise, any hydrogenation methods may be employed and any hydrogenation catalysts may be used, as are customary for the hydrogenation of aldehydes and are described in detail in the standard literature.

### EXAMPLES

### Example 1

### Hydrogenation of Hydroxypivaialdehyde to Neopentyl Glycol

### Hydrogenation Feed

1.1 mol of isobutyraldehyde were stirred with 1 mol of formaldehyde in the form of a 40% solution and 4 mol % of trimethylamine, based on isobutyraldehyde, at 75° C. for 1 h. The reaction solution was concentrated by distilling off low boilers, for example isobutyraldehyde, and a portion of the water at standard pressure. The resulting bottoms consisted of 75% by weight of hydroxypivaialdehyde, 20% by weight of water and approx. 5% by weight of other organic secondary components.

### Catalyst Preparation

All percentages reported under this subheading are, unless stated otherwise, percentages by weight. The percentage compositions reported are based on the oxidic constituents of the finished catalysts.

The feedstocks were a 20% by weight sodium carbonate solution and an aqueous solution I which comprised 2.67% by weight of Al and 5% by weight of Cu in the form of their nitrates.

In the precipitation, solution I and sodium carbonate solution were metered into a precipitation vessel at 80° C. such that a pH of 5.6 was established.
The precipitation mixture was transferred to a larger stirred vessel and adjusted there to a pH of 7.9 at 80° C. with sodium carbonate solution. The suspension was then passed onto a filter press.
The mixture was then filtered and washed with water to free it of nitrate. The filter paste was suspended in water and dried in a spray tower with hot air at outlet temperature 130-150° C. Thereafter, calcination is effected at a temperature of 375-390° C.
Subsequently, the powder was tableted with 3% by weight of graphite as an assistant to give 3×3 mm tablets.
The resulting tablets were then calcined in a heated rotary tube at a temperature of 600° C. over 60 min.

The catalyst consisted of 55% CuO and 45% by weight of Al₂O₃, and had a specific surface area (BET) of 95 m²/g and an Hg porosity of 0.38 ml/g with a tapped density of 1042 g/l.

150 ml of this Cu/Al₂O₃ catalyst were activated in a tubular reactor at 190° C. by passing over a mixture of 5% by volume of hydrogen and 95% by volume of nitrogen (total volume 50 l (STP)/h) at ambient pressure for 24 h.

### Hydrogenation

The starting solution used was the mixture described above as hydrogenation feed. From 0 to 7% by weight (based on the hydrogenation feed) of a 15% by weight aqueous solution of trimethylamine (from 2 to 5% by weight (based on the hydrogenation feed), or of a 5% by weight aqueous solution of citric acid in the comparative examples) were added to this mixture in order to establish the particular pH of the hydrogenation effluent specified in Table 1. The hydrogenation feed thus obtained was pumped over the catalyst in a hydrogenation reactor with liquid circulation (circulation:feed=10:1) with a catalyst hourly space velocity of 0.4 kg_{HPA}/I_{cat}×h in trickle mode at 40 bar and 120° C.

A comparison of the process according to the invention with comparative examples V1 and V2, in which the pH of the hydrogenation effluent is in each case outside the inventive range, is shown by Table 1.

For pH measurement, a Knick model 766 pH meter with a Schott N1041A glass electrode was used.

TABLE 1

| | pH of the | Conversion | NPG selectivity |
|---|---|---|---|
| Example | hydrogenation effluent | [%] | [%] |
| V1 | 6.4 | 84.8 | 98.4 |
| V2 | 6.9 | 91.9 | 99.2 |
| 1 | 7.8 | 97.1 | 99.9 |
| 2 | 8.3 | 97.2 | 99.7 |
| 3 | 8.6 | 97.6 | 99.6 |
| 4 | 8.9 | 97.9 | 99.3 |

NPG = neopentyl glycol

### Example 2

### Hydrogenation of Dimethylolbutanal (DMB) to Trimethylolpropane (TMP)

### Hydrogenation Feed

The hydrogenation feed was prepared in accordance with Example 6 of PCT/WO 98/28253.

### Catalyst Activation

300 ml of a Cu/TiO₂ catalyst J PCT/WO 99/44974 were activated in a tubular reactor at 190° C. by passing over a mixture of 5% by volume of hydrogen and 95% by volume of nitrogen (total volume 150 l (STP)/h) at ambient pressure for 24 h.

### Hydrogenation

The starting solution used was the mixture described above as hydrogenation feed. Between 0 and 3% by weight (based on the hydrogenation feed) of a 10% aqueous solution of citric acid were added to the mixture in order to establish the pH of the hydrogenation effluent specified in Table 2. The hydrogenation feed thus obtained was conducted through the reactor heated to 120° C. in trickle mode at H₂ pressure 80 bar. The hourly space velocity was 0.4 kg of dimethylolbutanal (DMB)/(I_{cat}*h). Some of the hydrogenation effluent was mixed again with the feed (circulation mode). The ratio of circulation to feed was 10:1. Table 2 shows averaged conversions and selectivities over several days at different ph. The pH was measured on samples of the reactor effluent at room temperature.

A comparison of the process according to the invention with comparative examples V3 and V4, in which the pH of the hydrogenation effluent was in each case outside the inventive range, is shown by Table 2.

For pH measurement, a Knick model 766 pH meter with a Schott N1041A glass electrode was used.

TABLE 2

| | pH of the | | |
|---|---|---|---|
| | hydrogenation | | |
| Example | effluent | Conversion | TMP selectivity |
| 5 | 7.4 | 98.9 | 95.7 |
| 6 | 7.2 | 97.8 | 94.0 |
| 7 | 7.1 | 97.2 | 93.4 |
| 8 | 7.0 | 95.4 | 92.6 |
| V3 | 5.1 | 83.8 | 92.3 |
| V4 | 6.2 | 81.2 | 92.8 |

TMP = Trimethylolpropane

1.-10. (canceled)

11. A process for catalytically hydrogenating a methylolalkanal of general formula (I) 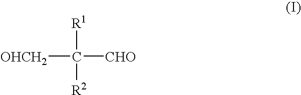
wherein
R¹ and R² are each independently a methylol group, an alkyl group having up to 22 carbon atoms, an aryl group having from 6 to 33 carbon atoms, or an aralkyl group having from 6 to 33 carbon atoms, and
said process comprises a hydrogenation feed and a hydrogenation effluent, comprising hydrogenating said methylolalkanal of general formula (I) in the liquid phase over a hydrogenation catalyst, and
maintaining a pH in the range of from 7.0 to 9.0 in said hydrogenation effluent by adding at least one tertiary amine, inorganic base., inorganic acid, or organic acid to said hydrogenation feed.

12. The process of claim 11, wherein said hydrogenation feed comprises less than 5% by weight of formaldehyde.

13. The process of claim 11, wherein said at least one tertiary amine is tri-n-alkylamine.

14. The process of claim 11, wherein said at least one tertiary amine is trimethylamine, triethylamine, tri-n-propylamine, and/or tri-n-butylamine.

15. The process of claim 11, wherein said at least one organic acid is acetic acid.

16. The process of claim 11, wherein said hydrogenation catalyst comprises at least one metal of transition groups 8 to 12 of the Periodic Table of the Elements.

17. The process of claim 11, wherein said hydrogenation catalyst comprises a supported catalyst.

18. The process of claim 17, wherein said supported catalyst comprises oxides of titanium, zirconium, hafnium, silicon, and/or aluminum.

19. The process of claim 17, wherein said hydrogenation catalyst comprises copper on an alumina or titania support material in the presence or absence of one or more of the elements magnesium, barium, zinc, or chromium.

20. The process of claim 11, wherein said methylolalkanal of general formula (I) is hydroxypivalaldehyde, pentaerythrose, or dimethyolbutanal.
